(19) United States
(12) Patent Application Publication
(10) Pub. No.: US 2004/0228786 A1
(43) Pub. Date: Nov. 18, 2004

(54) Oxidative purification of a flue gas containing oxygen and a combustible component

(76) Inventor: Walter Schicketanz, Frankenthal (DE)

Correspondence Address: Herbert B. Keil, KEIL & WEINKAUF, 1350 Connecticut Ave., N.W., Washington, DC, 20036, US

(21) Appl. No.: 10/765,983

(22) Filed: Jan. 29, 2004

(30) Foreign Application Priority Data
Feb. 3, 2003 (DE) 10304155.9

(51) Int. Cl. B01D 53/72; B01J 8/00; C07C 11/24

(52) U.S. Cl. 423/245.3

(57) ABSTRACT

A process for the oxidative purification of an exhaust gas containing oxygen and a combustible component by oxidative reaction in an oxidation reactor, in which the exhaust gas, before being introduced into the oxidation reactor, passes through a high-velocity path in which the flow velocity of the gas passing through is higher than the flashback velocity, and in which a substream of the flue gas liberated in the oxidative reaction is recirculated to the high-velocity path.

The present invention relates to a process for the oxidative purification of an exhaust gas containing oxygen and a combustible component by oxidative reaction in an oxidation rector, in which the exhaust gas, before being introduced into the oxidation reactor, passes through a high-velocity path in which the flow velocity of the gas passing through is higher than the flashback velocity.

Exhaust gases which contain a combustible component together with oxygen are formed in many different processes, for example oxidation processes in chemistry, coating processes, or in processes for dry cleaning. Owing to the simultaneous presence of an oxidizing agent (oxygen) and a combustible component, thermal purification of such exhaust gases, that is to say their combustion, requires special safety measures, in particular with respect to safely and reliably avoiding flashbacks.

An overview of oxidative processes for purifying exhaust gases, in particular of catalytic and thermal purification processes, may be found in J. M. Klobucar, Chem. Eng., February 2002, pages 62 to 67.

In catalytic exhaust gas purification, the exhaust gas is catalytically converted into more environmentally acceptable compounds at temperatures of typically from 200 to 650° C. in the presence of air and a catalyst. The use of catalysts makes far lower operating temperatures possible compared with pure combustion of the exhaust gas, which leads to advantages in the overall energy balance and choice of materials. The disadvantages of catalytic exhaust gas purification are closely connected to the use of catalysts. These usually contain noble metals, for example palladium or platinum, and therefore have a tendency, on contact with various compounds, to reversible or irreversible damage. If such compounds, termed catalyst poisons, are expected, generally a guard bed is provided upstream. Since the catalysts have only a limited service life, even in the absence of catalyst poisons, for reliable and long-lasting operation of a catalytic exhaust gas purification process, frequently the oxidation reactor must be constructed in duplicate. Furthermore, in the case of exhaust gases having a high content of combustible components, there is the risk of excessive reaction temperatures and flashback, and also the risk of damage to the catalyst and the plant.

In the thermal purification of exhaust gas, the exhaust gas is burnt at temperatures of typically from 800 to 1000° C. in the presence of air with or without what is called a supplemental fuel to form more environmentally friendly compounds, generally water and carbon dioxide. Generally, a differentiation is made between a direct flame oxidizer, a recuperative oxidizer and a regenerative oxidizer.

In the case of the direct flame oxidizer, the non-preheated exhaust gas to be purified is burnt with air in a flame which is generated by a supplemental fuel, for example natural gas or oil. To avoid flashback, generally at the inlet to the combustion chamber there is a high-velocity path in which the flow velocity of the exhaust gas fed is higher than the flashback velocity. A disadvantage of the direct flame oxidizer is the high consumption of supplemental fuel, in particular at low concentration of combustible components, since the exhaust gas to be burnt is fed in relatively cold and thus must be first brought to the desired combustion temperature with the aid of the heat of combustion of the supplemental fuel.

In the recuperative oxidizer, the non-preheated exhaust gas to be purified is preheated by the waste heat of the ideally autothermal combustion in the oxidizer and then burnt with air in the actual combustion chamber. The preheating generally takes place in such a manner that the exhaust gas fed, before entry into the combustion chamber, first flows through a heat exchanger which is operated on the other side with the hot flue gas. If the content of combustible components is not sufficient for autothermal combustion, the missing energy can be introduced by an auxiliary burner. Just said preheating makes possible substantially autothermal combustion, since the exhaust gas to be burnt already flows hot into the combustion chamber. However, precisely this also has a critical disadvantage. Since with increasing temperature in the exhaust gas its ignition performance also increases, there is the risk of flashback into the heat exchanger and thus the danger of relatively large damage. This danger is the more distinct, the higher the concentration of combustible components and the lower the flow velocity. Therefore, in particular in the case of exhaust gases having a high concentration of combustible components and/or great fluctuations in the composition and rate, the safe use of a recuperative oxidizer is not ensured.

In the regenerative oxidizer, the non-preheated exhaust gas to be purified is preheated via a hot heat storage medium and is burnt autothermally under ideal conditions in a subsequent combustion chamber. The hot flue gases are then passed over a second heat storage medium which is just then in the regenerative mode, and heat this up. If the first-mentioned heat storage medium has fallen in temperature to the extent that the desired combustion is no longer ensured, the flow is crossed over and the second heated heat storage medium is used for heating up. If the content of combustible components is not sufficient for the autothermal combustion, the missing energy can be introduced via an auxiliary burner. In the regenerative oxidizer also, substantially autothermal combustion is possible just via said preheating. As already described above in the case of the recuperative oxidizer, in the regenerative oxidizer there is also the danger with exhaust gases of high concentration of combustible components that the oxidation reaction will run away as soon as in the bed of the heat storage medium, that is to say will lead to an uncontrolled temperature increase which can damage the plant. There is also the danger of flashback into the heat exchanger and thus the danger of greater damage. Therefore, in the case of exhaust gases with a high concentration of combustible components and/or great fluctuations in the composition and rate, the safe use of a regenerative oxidizer is not ensured. In addition, the regenerative oxidizer, owing to its at least two heat storage chambers, each of which is designed to heat up the non-preheated exhaust gas, is very large and costly in terms of resources.

To prevent flashback safely, generally, in the exhaust gas feed to the oxidizer, a plurality of safety measures, which are independent of each other, are used, such as flame barriers and/or dilution of the exhaust gas. Surveys of this may be found, for example, in G.-G. Börger et al., VDI-Berichte No. 286, 1977, pages 131 to 134, in K. Schampel et al., Gas wärme international 27, 1978, November, pages 629 to 635, and in W. Hüning, Chem.-Ing.-Tech. 57, 1985, pages 850 to 857. Known flame barriers are, for example, liquid seals, flame arresters, screens, detonation arresters, velocity pathways, feeds of fresh air or flashback-proof nozzle feeds into the combustion chamber. The exhaust gas can be appropriately diluted, for example, with air. Thus, in the last-mentioned literature reference, in FIG. 5 there, is a combination of liquid seal, fresh air feed with velocity section, detonation arrester and flashback-proof nozzle feed into the combustion chamber. Although the use of a velocity path, when the required minimum velocity is maintained, does make reliable prevention of flashback possible, it does have the critical disadvantage that by feeding further air to maintain the required flow velocity, the total amount of exhaust gas increases and thus also the energy requirement for heating it up prior to combustion increases.

It is an object of the present invention, therefore, to find a process for the purification of an exhaust gas containing oxygen and a combustible component which does not have the abovementioned disadvantages, ensures safe long-term operation, is substantially autonomous from the energy point of view even when the exhaust gas produced markedly falls below the lower explosive limit and, in particular, also copes with changing exhaust gas rates and changing exhaust gas compositions without problem.

We have found that this object is achieved, accordingly, by a process for the oxidative purification of an exhaust gas containing oxygen and a combustible component by oxidative reaction in an oxidation reactor, in which the exhaust gas, before being introduced into the oxidation reactor, passes through a high-velocity path in which the flow velocity of the gas passing through is higher than the flashback velocity, which comprises recirculating to the high-velocity path a substream of the flue gas liberated in the oxidative reaction.

For the purposes of the present invention, oxidative purification is the oxidative reaction of an exhaust gas containing a combustible component with oxygen. The oxidative reaction can be performed both catalytically and non-catalytically. A non-catalytic reaction is combustion, which is generally also termed thermal reaction. In the oxidative purification, the exhaust gas fed is converted into predominantly more environmentally friendly compounds. If the exhaust gas contains, as combustible components, only hydrogen-, carbon- and/or oxygen-containing compounds, these are generally reacted to form water and carbon dioxide. If the exhaust gas, in addition, contains further elements, for example chlorine or sulfur, these are converted into more stable compounds of chlorine or sulfur, for example hydrogen chloride, chlorine oxides or sulfur oxides. The gas obtained by the oxidative reaction is termed flue gas.

Of importance in the inventive process is the use of a high-velocity path before introduction of the exhaust gas into the oxidation reactor, in which the flow velocity of the gas flowing through is higher than the flashback velocity, and to which a substream of the flue gas being released in the oxidative reaction is recirculated.

As high-velocity paths which can be used in the inventive process, in principle all apparatuses can be used through which the exhaust gas can flow together with the recirculated flue gas at the flow velocity required to prevent flashback. The high-velocity path can be situated, for example, as a separate apparatus in the exhaust gas feed line to the oxidation reactor, or else be situated directly at the inlet of the oxidation reactor, for example in the form of one or more nozzles. Furthermore, for the purposes of the present invention, a high-velocity path is also a high-velocity valve, as described, for example, in the standard DIN EN 12874:2001 under point 3.1.18. Generally they comprise one-or more tubes having an inner hydraulic diameter of generally from 5 to 50 mm, preferably from 5 to 30 mm. Their length is usually from 1 to 5 m, and preferably from 0.5 to 2 m. The high-velocity path, in the direction of flow, is always upstream of the plant part in which the oxidation reaction takes place. Advantageously, the paths through which flow is to pass between the high-velocity path and the plant part in which the oxidation reaction takes place should be as short as possible.

For the purposes of the present invention, the flow velocity of the gas flowing through the high-velocity path is the mean gas velocity in the individual pipes or channels of the high-velocity path, the flow velocity being based on the gas flowing through the high-velocity path under the conditions (temperature, pressure) occurring there. The flow velocity can be measured directly in the high-velocity path or else upstream or downstream of the high-velocity path, in both last-mentioned cases, a corresponding conversion needing to be carried out incorporating the individual cross sections and any differences in temperature and pressure. Generally, the flow velocity is measured directly upstream of the high-velocity path. Instruments which can be used to measure the flow velocity are instruments generally known to those skilled in the art, in which case obviously attention needs to be paid to appropriate stability to the temperature present and the medium flowing through. Examples of suitable instruments are differential pressure instruments, velocity and mass flow meters, in particular Pitot, Prandtl, target, vortex, ultrasound and heat-wire instruments, as are described, for example, in J. W. Dolenc, Chem. Eng. Prog., January 1996, pages 22 to 32. To ensure correct measurement of the flow velocity, in many cases it is advantageous to use what is termed a flow conditioner upstream of the actual metering instrument, in particular in the cases where, owing to the inner diameter of the pipe and the flow velocity, a corresponding proportion of turbulent flow is to be assumed.

For the purposes of the present invention, the flashback velocity is the flow velocity of the gas flowing through at which, in the piping piece under consideration, assuming that a stable ignition flame were to exist at the end of this piping piece, flashback would just occur. In the context of the present invention, the piping piece under consideration is the high-velocity path.

DIN EN 12874:2001, in section 9.2, gives a measurement protocol by which the flashback velocity can be determined experimentally. For this, a test apparatus set up according to FIG. 6 of the cited standard containing the corresponding high-velocity path is used as piping piece to be measured. At the end of the high-velocity path, as shown in FIG. 6, there is a stabilized ignition flame. The exhaust gas to be measured, or a gas mixture corresponding to this composition, is then passed through the high-velocity path at the desired operating temperature. The procedure starts with a flow velocity which is higher than the flashback velocity and the velocity is then reduced until a flashback occurs. The measurement shall be repeated three times in accordance with the cited standard and the highest flow velocity which led to a flashback is to be recorded as flashback velocity.

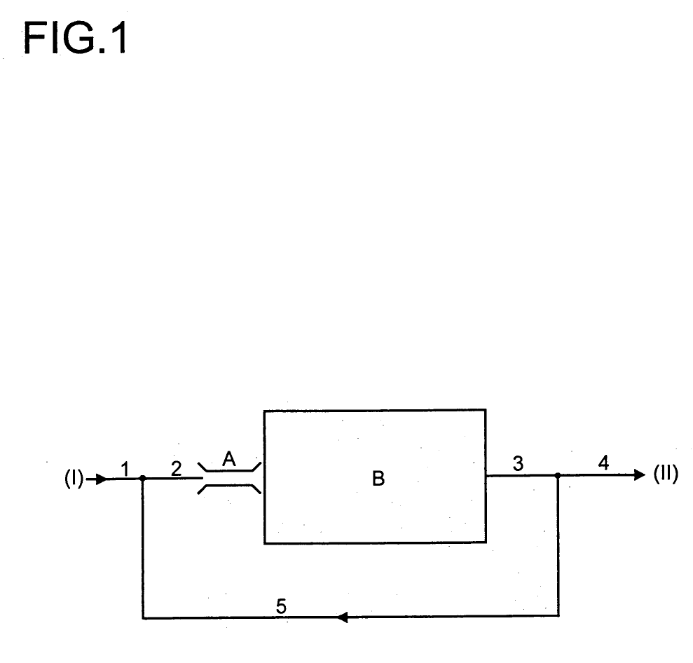

FIG. 1 shows a block diagram of the inventive process. Exhaust gas (I) is passed via line (1) and (2) to the high-velocity path (A) in which the flow velocity of the gas flowing through is higher than the flashback velocity, and after passing through the high-velocity path (A), exhaust gas is passed into the oxidation reactor (B). There it is oxidatively reacted to form the flue gas which leaves the apparatus for oxidative purification as flue gas (II) via line (3) and (4). Via line (5), a substream of the flue gas is recirculated to the high-velocity path (A).

In the inventive process, the recirculation of flue gas in the high-velocity path establishes a flow velocity which is preferably at least 1.2 times, particularly preferably at least 1.5 times, and very particularly preferably at least 2 times, the flashback velocity.

The desired flow velocity in the high-velocity path is preferably established via recirculation of the flue gas under control of volumetric flow rate, the flow velocity in the high-velocity path being used as control variable. By monitoring the flow velocity and the associated recirculation of the flue gas under control of volumetric flow rate to achieve the preset value, a particularly reliable process is ensured, since as a result fluctuations in the exhaust gas rate can be compensated for. The preset value of the flow velocity to be set is generally determined using the abovementioned value of the flashback velocity.

In the case of exhaust gases which continuously, or intermittently, have relatively high concentrations of combustible constituents, in order to keep the temperature in the oxidation reactor below a defined value, or within a defined range, the flow velocity in the velocity path can also be increased beyond said multiple. Likewise, alternatively or additionally, flue gas or air can be introduced into the oxidation reactor directly, that is to say without passing it via the high-velocity path.

To promote the oxidative reaction in the oxidation reactor, in particular it is advantageous in the case of exhaust gases of low concentration of combustible components to introduce previously preheated exhaust gas into the oxidation reactor. As a result, because of the lower difference between the temperature of the preheated exhaust gas to be introduced and the oxidation temperature, less heat is removed from the oxidation reactor and thus the oxidative reaction is promoted. It is particularly advantageous, in the inventive process, to heat the exhaust gas, upstream of the high-velocity path, via a heat exchanger operated by the flue gas waste heat. The flue gas waste heat can follow here directly in the form of a heat exchanger operated by the flue gas or indirectly, for example by producing steam and preheating the exhaust gas via a heat exchanger operated by this steam. By means of the above-described targeted setting of the flow velocity in the high-velocity path, safe operation is also ensured when preheated exhaust gas is fed.

In the abovementioned preheating of the exhaust gas, it must be noted that the lower explosive limit is temperature-dependent and thus a mixture which is not explosive at low temperature can pass into the explosive range via preheating. In this case, even the very smallest ignition sources would cause an explosion in the relevant plant part. In the inventive process, therefore, for safety considerations, it should be ensured that the explosive range is avoided. Therefore, the temperature of the preheated exhaust gas should be below the temperature of the lower explosive limit. For the purposes of the present invention, lower explosive limit is the relevant explosive limit under the existing pressure and the existing gas composition.

When an exhaust gas is used which is constant with respect to its composition, it is generally sufficient to determine the temperature of the lower explosive limit experimentally or by calculation in advance and, on the basis of this value, to establish the maximum temperature for the preheated exhaust gas.

When an exhaust gas which is varying with respect to its composition is used, for considerations of safety, it is advantageous to monitor the temperature of the lower explosive limit continuously or intermittently. Suitable instruments for monitoring the lower explosive limit are, for example, gas detectors. In gas detectors, for example, a sample of the gas to be measured is burnt catalytically and, via the heat of reaction generated, the current concentration of combustible gases is calculated. From the current concentration, the corresponding temperature then follows from the relationship, to be determined in advance, between the lower explosive limit and the concentration of combustible gases. The signal from the gas detector can, in addition to the described monitoring of the oxidative purification of the exhaust gas, for example, also be used to control the upstream process steps and, for example, activate safety circuits.

In a preferred variant of the inventive process, the concentration of combustible gases is measured upstream of the heat exchanger, that is to say in the non-preheated exhaust gas. On the basis of the lower explosive limit temperature determined therefrom, the desired exhaust gas temperature to be set is then established and the heat exchanger operated appropriately. If, in addition, it is also ensured that the flow time for the exhaust gas between the analysis point and the heat exchanger is greater than the overall response time of said controller, safe operation can be ensured even in the event of large variations in the composition of the exhaust gas.

Preferably, in the inventive process, the temperature of the preheated exhaust gas is set in such a manner that the lower explosive limit, at the existing pressure and existing gas composition, is at least 4/3 times, particularly preferably at least 2 times and, very particularly preferably, at least 4 times the value of the existing concentration of combustible components. Said settings are distinguished by particularly high safety margins.

If the exhaust gas originates from a heterogeneously catalyzed gas-phase oxidation, it must be assumed that the lower explosive limit of the resultant reactor discharge is at a temperature above the hottest temperature of the heterogeneously catalyzed gas-phase oxidation. Since the product of value formed is usually removed from the resultant reactor discharge, what remains as exhaust gas is a gas which has a markedly lower concentration of combustible components than the resultant reactor discharge. Thus the lower explosive limit of this exhaust gas is even at a still higher temperature than that of the resultant reactor discharge. Therefore, when an exhaust gas is used which originates from a heterogeneously catalyzed gas-phase oxidation, it is particularly advantageous to set the temperature of the preheated exhaust gas in such a manner that it corresponds at maximum to the hottest temperature of the heterogeneously catalyzed gas-phase oxidation. This variant, even without continuous or intermittent analysis of the exhaust gas, offers very high safety. Since heterogeneously catalyzed gas-phase oxidations are generally carried out at temperatures in the range from 300 to 600° C., thus significant preheating of the exhaust gas is already possible without the expenditure of continuous or intermittent analysis with control.

Oxidation reactors which can be used in the inventive process are oxidation reactors operating not only catalytically, but also non-catalytically.

In a catalytic oxidation reactor, the oxidative reaction takes place on a heterogeneous catalyst which is situated in a catalyst bed through which the exhaust gas to be oxidized is passed. The catalysts used can generally be the catalysts which are known to those skilled in the art for oxidative purification of exhaust gases. Suitable examples are palladium and/or platinum on a support, for instance aluminum oxide. The catalytic oxidative reaction generally takes place at a temperature in the range from 200 to 650° C.

In a non-catalytic oxidation reactor, the oxidative reaction generally takes place thermally, that is to say by combustion, at a temperature of usually from 700 to 1200° C. The combustion takes place in a combustion chamber into which the exhaust gas to be burnt is introduced. At an appropriately high content of combustible components and/or an appropriately high temperature of the exhaust gas fed, autothermal combustion may be possible. Autothermal combustion is distinguished by the required fuel originating solely from the exhaust gas to be burnt. If the content of combustible components and/or the temperature of the exhaust gas is correspondingly low, the use of an auxiliary or supplemental burner can be necessary. This is operated with an additional fuel, for example natural gas or oil, and supplies the remaining energy required for the combustion. Generally, combustion chambers which are operated autothermally also contain a supplementary burner in order, in particular, to make it possible to start up the plant and, in the event of fluctuations or interferences with the exhaust gas feed, to ensure continuous combustion.

If a non-catalytic oxidation reactor is used, it may be advantageous if the upstream high-velocity path is situated directly upstream of the inlet into the combustion chamber, or is integrated into the burner head. Possible designs for this are described, for example, in G.-G. Börger et al., VDI-Berichte No. 286, 1977, pages 131 to 134, and in particular FIG. 5.

Preferably, in the inventive process, a combustion chamber is used as oxidation reactor.

Depending on requirements, it is possible in the inventive process to connect further, upstream of the actual oxidation reactor, an apparatus for heating up the exhaust gas fed. Suitable apparatuses are, for example, flue gas-operated heat exchangers, or regenerative heat-storage media, as described, for instance, in J. M. Klobucar, Chem. Eng., February 2002, pages 62 to 67.

Despite the abovementioned measures, in order to virtually exclude flashback, for further safety, in the inventive process, in the exhaust gas feed, generally one or more further safety measures against flashback are used. Suitable measures are flashback preventers, such as liquid seals, flame arresters, screens, detonation safeguards and measures such as flashback-free nozzle feed into the combustion chamber. They are described, for example, in G.-G. Börger et al., VDI-Berichte No. 286, 1977, pages 131 to 134, in K. Schampel et al., Gas wärme international 27, 1978, November, pages 629 to 635 and in W. Hüning, Chem.-Ing.-Tech. 57, 1985, pages 850 to 857. These safety measures can be situated upstream and/or downstream of the high-velocity path; preferably they are upstream of the high-velocity path, in the direction of flow.

In a preferred variant of the inventive process, the hot flue gas formed is utilized energetically for heating up external energy carriers. Energetic utilization, for the purposes of the present invention, is, in particular, the production of hot water, steam and superheated steam. The corresponding processes for the energetic utilization of the flue gas and the apparatuses required therefor are generally known to those skilled in the art.

In a particularly preferred variant of the inventive process, the desired temperature of the flue gas to be recirculated is set by mixing flue gas of two different temperatures, the difference in the flue gas temperatures being obtained via the energetic utilization in the form of an intermediate heat exchanger.

The exhaust gas to be used in the inventive process can originate from the most varied sources in which a gas to be disposed of is formed which contains oxygen and a combustible component. Examples of possible sources are chemical processes, in particular oxidation reactions, working processes, such as painting or dry cleaning, and also natural sources.

Combustible components in the exhaust gas which come into consideration in the inventive process are in principle all inorganic or organic compounds which are oxidizable by oxygen and which are gaseous under the existing conditions. These can be a single compound or a mixture of different compounds. Suitable combustible components are, for example, hydrogen, aliphatic, aromatic or araliphatic hydrocarbons, alcohols, aldehydes, ketones, carboxylic acids, ammonia or amines. Generally, the exhaust gas to be disposed of contains from 0.01 to 10% by volume, preferably from 0.01 to 5% by volume, and particularly preferably from 0.1 to 2% by volume, of combustible components.

Preferably, the inventive process is used for purifying exhaust gases which originate from the heterogeneously catalyzed gas-phase oxidation of n-butane and/or n-butenes to maleic anhydride, of o-xylene to phthalic anhydride, of propene to acrylic acid, of isobutene to methacrylic acid, of 1,2-ethanediol to glyoxal, of ethene to ethylene oxide, of propene to acrolein, of propene and ammonia to acrylonitrile, of olefins to aldehydes or ketones, of methanol to formaldehyde and/or of methane and ammonia to prussic acid, and particularly preferably of n-butane and/or n-butenes to maleic anhydride, of o-xylene to phthalic anhydride, of propene to acrylic acid, of isobutene to methacrylic acid, of 1,2-ethanediol to glyoxal and/or of ethene to ethylene oxide.

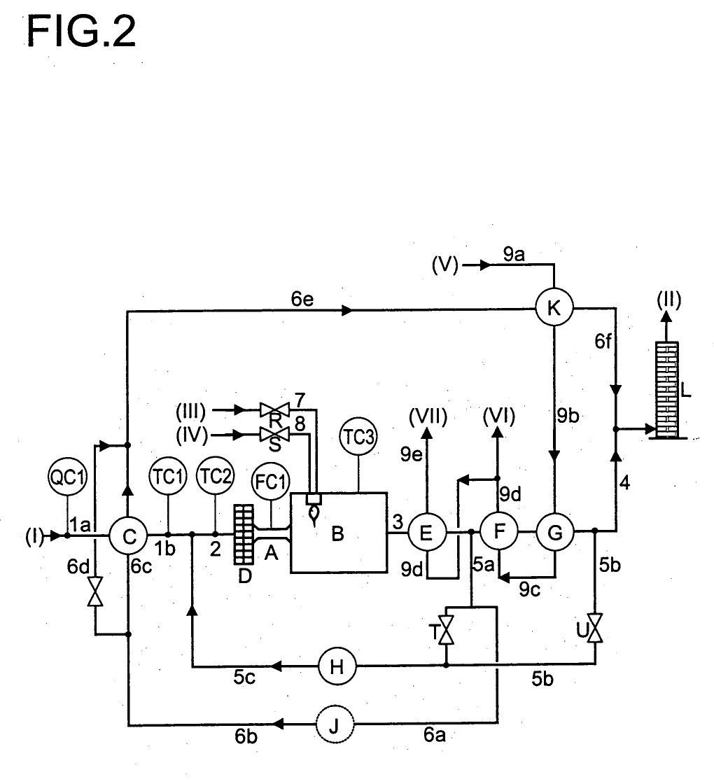

The simplified process flowchart of a preferred embodiment is shown in FIG. 2. The apparatuses and valves are provided with capital letters and are described in more detail in the description. The lines are numbered throughout in arabic numbers. The inputs and outputs of material streams are numbered in roman numerals and are likewise described in more detail in the description. The control and instrumentation systems bear the usual nomenclature with a letter suffix for serial numbering: “Q” is analytical measurement, “T” is temperature measurement, “F” is flow measurement and “C” for control circuit.

The exhaust gas (I) is fed via line (1a), the heat exchanger (C), in which the exhaust gas is preheated by flue gas passing through, and line (1b). Via line (5c), recirculated flue gas is added and is fed together with the freshly supplied exhaust gas from line (1b) via line (2) and a static flashback preventer (D), preferably a screen, to the high-velocity path (A) in which the flow velocity of the gas passing through is higher than the flashback velocity. Advantageously, the exhaust gas and the recirculated flue gas are mixed upstream of the high-velocity path, for example by using a static mixer or substream mixing. Downstream of the high-velocity path (A), the gas is introduced via one or more burners (now shown) into the combustion chamber (B). This, in particular for start up and for using exhaust gases which are not burnable autothermally, contains an auxiliary burner which can be present as a separate burner or integrated into the burner or the burners and which, when required, can be operated via line (7) with air (III) and via line (8) with fuel (IV), for example natural gas. In the combustion chamber in which further burners for other substances and exhaust gases can also be integrated, the exhaust gas is oxidatively reacted to give the flue gas. This leaves the combustion chamber (B) in a hot state and, via line (3), is passed to a number of serial-connected heat exchangers. In heat exchanger (E), superheated steam (VII) is produced, in heat exchange (F) saturated steam is produced, and in heat exchanger (G) warm water (feed water preheating) is produced. The energetically utilized and cooled flue gas passes via line (4) to the stack (L) and is discharged into the atmosphere as flue gas (II). It may be noted at this point that in the case of a pollutant-containing flue gas, for instance sulfur oxides or chlorine compounds, further diverse purification apparatuses can be connected in an intermediate position upstream of the emission into the atmosphere. The portion of the flue gas to be recirculated is taken off via the lines (5a) and (5b). The heat exchangers connected in an intermediate position ensure a corresponding temperature difference between the flue gas in line (5a) and that in line (5b), so that via suitable mixing it is possible to set the temperature in a broad range. The flue gas to be recirculated is then passed via line (5c) and via a recirculation fan (H) to the high-velocity path (A). The portion of the flue gas which is used for operating the heat exchanger (C) is likewise taken off via line (5a), and via line (6a), an optional fan (J), line (6b) and line (6c), is fed to the heat exchanger (C). For rapid control of the temperature in the heat exchanger (C), this has a bypass via line (6d). The flue gas can then be passed via line (6e) to a heat exchanger (K) for preheating the feed water (V), and line (6f), likewise to the stack (L). The energetic utilization of the hot flue gas takes place as shown by way of example in FIG. 2, by generating steam (VI) and/or superheated steam (VII), with use of the above-mentioned heat exchangers (K), (G), (F) and (E).

It may be mentioned, that depending on the design, the heat exchanger (E), for example, can also be integrated in the combustion chamber, so that line (3) would virtually be omitted.

Below, the control cirucits of the preferred embodiment are described in more detail.

For better clarity, in FIG. 2, the control lines are omitted. “TC1” measures the temperature of the exhaust gas directly downstream of the heat exchanger (C). This measured value serves for temperature monitoring of the preheated exhaust gas and, via valves (P) and (O), controls the temperature of the preheated exhaust gas. “TC2” measures the temperature of the exhaust gas mixed with the recirculated flue gas and can optionally be set via the valves (T) and (U) for fine control of the temperature in the high-velocity path (A). “FC1” measures the flow velocity in the high-velocity path (A) and serves for controlling the volumetric flow rate of recirculated flue gas via the valves (T) and (U). “TC3” measures the temperature in the combustion chamber (B) and, as required, closes the auxiliary burner via the valves (R) and (S), or controls it. “QC1” measures the concentration of combustible components in the exhaust gas and thus gives a parameter from which the temperature of the lower explosive limit can be determined. In accordance with the desired safety margin, care must be taken that the temperature downstream of the heat exchanger (C) corresponds at maximum to the desirable permissible temperature. The mean value of “QC1” can preset directly the maximum temperature of the preheated exhaust gas. If the concentration of combustible components in the exhaust gas increases very rapidly to a high value, a safety circuit is also possible via “QC1”. This would then increase, for example, the amount of recirculated flue gas by accelerating the fan (H) and/or by opening valve (U) and/or shut off the heat exchanger (C) by closing valve (O). If required, switching over the heat exchanger (C) to a cooling mode, which can be operated, for example, with water, is also conceivable.

If the temperature in the combustion chamber (B) reaches the upper limit of the desired temperature range, then, for cooling, the feed of additional (ambient) air into the combustion chamber can be triggered via “TC3”. This can be fed, for example, via valve (R) and line (7) or via an additional apparatus which is not shown in FIG. 2. Furthemore, it is also possible also to induce, via “TC3”, adequate recirculation of cold flue gas, for example, via valve (U) and the fan (H). The recirculation of cold flue gas to control the temperature in the combustion chamber comes into effect, in particular, when the exhaust gas has a high content of combustible components and thus its energy content is therefore relatively high.

The inventive process makes possible the oxidative purification of an exhaust gas containing oxygen and a combustible component, ensuring safe long-term operation. The inventive process, from the energetic aspect, even when conditions fall markedly below the lower explosive limit of the exhaust gas produced is substantially autonomous and copes, in particular, even with changing exhaust gas rates and changing exhaust gas compositions without problem. The substantial energy independence of the inventive process is particularly advantageously achieved by preheating the exhaust gas which is made possible by the use of the high-velocity path and ensuring that the flow velocity of the gas flowing through is higher than the flashback velocity. The preferred control of flow velocity by recirculated flue gas makes possible automatic adaptation of the process to fluctuating exhaust gas rates and fluctuating concentrations of combustible components. The specific recirculation of flue gas in addition avoids the use of foreign gases, for example air.

Furthermore, the invention relates to an apparatus for the oxidative purification of an exhaust gas containing oxygen and a combustible component according to the inventive process comprising

a) a feed line (1) for the exhaust gas (I);

b) a oxidation reactor (B);

c) a high-velocity path (A) which is situated between the feed line (1) and the exhaust-gas side of the oxidation reactor (B);

d) a line (5) for recirculating flue gas, which line is situated between the flue-gas side of the oxidation reactor (B) and the exhaust-gas side of the high-velocity path (A); and

e) a removal line (4) for the flue gas (II).

A corresponding block diagram is given in FIG. 1.

1. A process for the oxidative purification of an exhaust gas containing oxygen and a combustible component by oxidative reaction in an oxidation reactor, in which the exhaust gas, before being introduced into the oxidation reactor, passes through a high-velocity path in which the flow velocity of the gas passing through is higher than the flashback velocity, which comprises recirculating to the high-velocity path a substream of the flue gas liberated in the oxidative reaction.

2. A process as claimed in claim 1, wherein the recirculation of flue gas in the high-velocity path establishes a flow velocity which is at least 1.2 times the flashback velocity.

3. A process as claimed in claim 1, wherein the flue gases are recirculated to the high-velocity path under control of volumetric flow rate and the flow velocity in the high-velocity path is used as control variable.

4. A process as claimed in claim 1, wherein the exhaust gas, upstream of the high-velocity path, is preheated via a heat exchanger operated by the flue gas waste heat.

5. A process as claimed in claim 1, wherein the temperature of the preheated exhaust gas is set in such a manner that the lower explosive limit, at the existing pressure and existing gas composition, is at least 4/3 times the value of the existing concentration of combustible components.

6. A process as claimed in claim 1, characterized in that an exhaust gas is used which originates from a heterogeneously catalyzed gas-phase oxidation and the temperature of the preheated exhaust gas is set in such a manner that it corresponds at maximum to the hottest temperature of the heterogeneously catalyzed gas-phase oxidation.

7. A process as claimed in claim 1, wherein the oxidation reactor used is a combustion chamber.

8. A process as claimed in claim 1, wherein an exhaust gas is used which originates from the heterogeneously catalyzed gas-phase oxidation of n-butane and/or n-butenes to maleic anhydride, of o-xylene to phthalic anhydride, of propene to acrylic acid, of isobutene to methacrylic acid, of 1,2-ethanediol to glyoxal and/or of ethene to ethylene oxide.

9. An apparatus for the oxidative purification of an exhaust gas containing oxygen and a combustible component according to the process as claimed in claim 1 comprising
a) a feed line (1) for the exhaust gas (I);
b) an oxidation reactor (B);
c) a high-velocity path (A) which is situated between the feed line (1) and the exhaust-gas side of the oxidation reactor (B);
d) a line (5) for recirculating flue gas, which line is situated between the flue-gas side of the oxidation reactor (B) and the exhaust-gas side of the high-velocity path (A); and
e) a removal line (4) for the flue gas (II).
